(19) United States
(12) Patent Application Publication
(10) Pub. No.: US 2019/0224186 A1
(43) Pub. Date: Jul. 25, 2019

(54) TREATMENT OF MULTIPLE SCLEROSIS WITH CHS-131

(71) Applicant: InteKrin Therapeutics, Inc., Redwood City, CA (US)

(72) Inventors: Barbara Finck, San Francisco, CA (US); Robert Zivadinov, Orchard Park, NY (US); Hong Tang, Belmont, CA (US)

(21) Appl. No.: 16/333,171

(22) PCT Filed: Sep. 13, 2017
(86) PCT No.: PCT/US17/51414

§ 371 Date: Mar. 13, 2019

Related U.S. Application Data
(60) Provisional application No. 62/394,046, filed on Sep. 13, 2016.
(60) Provisional application No. 62/436,356, filed on Dec. 19, 2016.
(60) Provisional application No. 62/460,868, filed on Feb. 19, 2017.
(60) Provisional application No. 62/491,071, filed on Apr. 27, 2017.

(51) Int. Cl. A61K 31/47; A61P 25/28

(52) U.S. Cl. CPC A61K 31/47; A61P 25/28

(57) ABSTRACT

Methods of treatment of multiple sclerosis (MS) in humans, and in women in particular, comprising administering CHS-131 of the following formula: (I) or a pharmaceutically acceptable salt, prodrug or isomer of CHS-131.

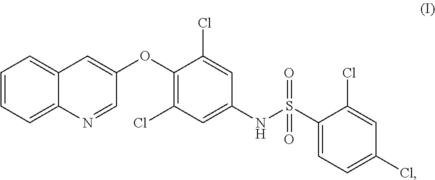 [SMILES: O=S(=O)(Nc1cc(Cl)c(Oc2cnc3ccccc3c2)c(Cl)c1)c1ccc(Cl)cc1Cl]

### BACKGROUND OF THE INVENTION

Multiple sclerosis or MS is a disease that affects the brain and spinal cord resulting in loss of muscle control, vision, balance, sensation (such as numbness) or thinking ability.

In MS, parts of the brain and spinal cord, which together form the central nervous system or CNS are recognized as being foreign and are attacked by one's own immune system. At the cellular level, the CNS is made up by neurons, the “thinking cells” of the nervous system, and glial cells, which perform a wide variety of vital functions. The cell bodies of the neurons are connected to one-another by axons, which function like wires tying neuronal networks together. There are billions of axons in the CNS, which, like copper wires, need to be insulated to prevent loss of signaling, to boost the speed of signaling and to prevent signal interference. The insulating material of the CNS, called myelin, is a specialized organelle of glial cells, which wrap the myelin around the axons. In MS, elements of myelin are recognized as foreign, and are attacked by the individual's own immune system. As a result of these immune attacks the myelin is destroyed, and often, the associated axons are also damaged leading to death. This is an iterative process broken up by periods of remyelination. However, while myelin can reform, eventually the pool of cells that can make myelin is depleted, resulting in areas of chronic CNS demyelination that eventually form scars, also known as plaques, and whose formation is known as sclerosis. When this process of sclerosis is iterative, the resulting form of MS is called relapsing/remitting MS. There is also another rarer form of MS, called primary progressive MS, where no remission occurs. In either case, without the myelin, electrical signals transmitted throughout the brain and spinal cord are disrupted or halted. The affected areas of the brain then become unable to properly send and to receive messages. It is this breakdown of communication that causes the symptoms of MS.

There are a variety of medications available that can reduce the frequency and severity of MS symptoms in some people with MS. Symptoms may be divided into three categories: primary, secondary, and tertiary. Primary symptoms are a direct result of the demyelination process. This impairs the transmission of electrical signals to muscles (to allow them to move appropriately) and the organs of the body (allowing them to perform normal functions.) The symptoms include weakness, tremors, tingling, numbness, loss of balance, vision impairment, paralysis, and bladder and bowel problems.

Secondary symptoms result from primary symptoms. For example, paralysis (a primary symptom) can lead to bedsores (pressure sores) and bladder or urinary incontinence problems can cause frequent, recurring urinary tract infections. These symptoms can be treated, but the ideal goal is to avail them by treating the primary symptoms.

Tertiary symptoms are the social, psychological, and vocational complications associated with the primary and secondary symptoms. Depression, for example, is a common problem among people with MS.

The course of multiple sclerosis is highly variable. In particular, the earliest stages of the disease can be somewhat unpredictable. Because of this uncertainty, doctors often tell their patients that they “probably” or “possibly” have MS. Diagnosis is based on the combination of clinical presentations, findings on magnetic resonance imaging (“MM”) and other tests, and patterns of recurrence. At present, there is no way to predict how each person's disease will progress. It often takes an extended period of time before a definitive diagnosis of MS can be made. There are three main courses that MS takes:

Relapsing-remitting MS (“RRMS”), which is characterized by unpredictable acute attacks, called “exacerbations,” with worsening of symptoms followed by full, partial or no recovery of some function. These attacks appear to evolve over several days to weeks. Recovery from an attack takes weeks sometimes months. The disease does not worsen in the periods between the attacks. This pattern usually occurs early in the course of MS in most people;

Primary-progressive MS, which is characterized by a steady progression of disability, without any obvious relapses and remissions. This form of disease occurs in just 15% of all people with MS, but is more common in people who develop the disease after the age of 40; and

Secondary-progressive MS, which initially begins with a relapsing-remitting course, but later evolves into progressive disease. The progressive part of the disease may begin shortly after the onset of MS, or it may occur years to decades later.

A true exacerbation of MS is caused by an area of inflammation (i.e. swelling) in the nerves of the brain and spinal cord system followed by something called demyelination, which is the destruction of myelin. The myelin is the fatty sheath that surrounds and protects the nerve fibers. An exacerbation of MS may be mild and not cause a noticeable impairment in functioning or may significantly interfere with a person's daily life. Untreated, exacerbations can last from several days to several weeks, although they may extend into months.

Cortical atrophy, or loss of volume, is known to be a crucial component of multiple sclerosis. Atrophy is present at early stages of the disease. Volume loss is not thought to be simply due to water loss and subsequent shrinkage of the tissue, but due to the loss of functional tissue. Thus, it is associated with clinical (especially cognitive) dysfunction in patients with MS. Since the underlying mechanisms of MS are still unknown, treatments are necessary that slow, stop, or reverse the loss of cortical volume and also slow, stop, or reverse the clinical dysfunction that results from MS.

Several methods are used to diagnose and determine the impact of multiple sclerosis on patients. The McDonald criteria are used to diagnose multiple sclerosis on clinical grounds and MRI lesions consistent with MS. The Expanded Disability Status Scale (EDSS) quantifies disability in eight Functional Systems (FS) and allows neurologists to assign a Functional System Score (FSS) in each of these. The Multiple Sclerosis Functional Composite (MSFC) is a three-part, standardized, quantitative, assessment of function. The three components of the MSFC measure leg function/ambulation, arm/hand function, and cognitive function. The EDSS and MSFC scores are useful to determine if a drug improves or prevents loss of cognitive or physical function of a patient with MS. A drug is said to reduce MS-related dysfunction if it reduces a patient's EDSS or MSFC score.

CHS-131 (also known as INT-131) is a novel, first-in-class, selective modulator of PPARγ which crosses the blood-brain barrier and exerts potent anti-inflammatory effects in the central nervous system without evidence of systemic immunosuppression. CHS-131 has been studied in over 600 patients in multiple indications and has been shown to improve clinical and neuropathological outcomes in animal models of experimental autoimmune encephalomyelitis.

CHS-131 is structurally different from other PPARγ agonists. CHS-131 lacks the TZD (glitazone) scaffold of rosiglitazone and pioglitazone. Therefore, CHS-131 binds the AF2 (transcriptional activation function 2) helix without contacting helix 12. As a result, CHS-131 selectively activates PPARγ functions.

PPARγ protein function regulates target gene transcription in a ligand-dependent, cofactor-dependent manner by differential co-factor/co-repressor recruitment. As a result of these complex combinatorial chemistry mechanisms, and the unique structure of CHS-131, the effects of selective activation of PPARγ is difficult to predict. For instance, it has been shown that subjects who are administered CHS-131 lack TZD-induced adverse events. Therefore, transcriptional activation effected by CHS-131 differs from other PPARγ agonists. As a result, it cannot be assumed that CHS-131 will have the effect on patients as other PPARγ agonists.

While the CHS-131 compound was previously proposed for the treatment of MS (See, U.S. Pat. No. 9,061,020), it remains important to determine if CHS-131 can reduce the loss of cortical volume and if CHS-131 can improve or prevent the loss of cognitive or physical function of a patient with MS.

### SUMMARY OF THE INVENTION

The present invention is directed to a method of reducing cortical atrophy in a subject suffering from multiple sclerosis comprising administering to the subject, at regular dosing intervals, a pharmaceutical composition comprising a therapeutically effective amount of a compound of formula (I),

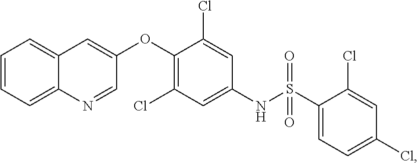 [SMILES: O=S(=O)(Nc1cc(Cl)c(Oc2cnc3ccccc3c2)c(Cl)c1)c1ccc(Cl)cc1Cl]

or a pharmaceutically acceptable salt, prodrug, or isomer thereof.

In one embodiment, the pharmaceutical composition is administered to the subject daily and the therapeutically effective amount of the compound is about 3 milligrams.

The present invention is further directed to a method of reducing loss of cortical volume in a subject suffering from multiple sclerosis comprising administering to the subject, at regular dosing intervals, a pharmaceutical composition comprising a therapeutically effective amount of a compound of formula (I), or a pharmaceutically acceptable salt, prodrug, or isomer thereof.

In one embodiment, the pharmaceutical composition is administered to the subject daily and the therapeutically effective amount of the compound is about 3 milligrams.

The present invention is further directed to a method of treating multiple sclerosis in a woman comprising administering to a woman at regular dosing intervals a pharmaceutical composition comprising a therapeutically effective amount of a compound of formula (I),

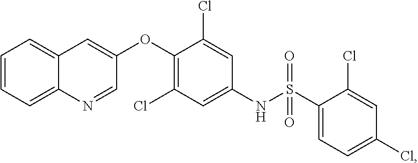 [SMILES: O=S(=O)(Nc1cc(Cl)c(Oc2cnc3ccccc3c2)c(Cl)c1)c1ccc(Cl)cc1Cl]

or a pharmaceutically acceptable salt, prodrug, or isomer thereof.

In one embodiment, the pharmaceutical composition is administered to the woman daily and the therapeutically effective amount of the compound is about 5 milligrams.

In one embodiment, the method provides a reduction in number of new gadolinium CE T1-weighted lesions in the woman over six months by at least about 45%, at least about 50%, at least about 60%, at least about 65%, at least about 70%, or at least about 80%.

The present invention is further directed to a method of treating multiple sclerosis in a subject comprising administering to the subject, at regular dosing intervals, a pharmaceutical composition comprising a therapeutically effective amount of a compound of formula (I), or a pharmaceutically acceptable salt, prodrug, or isomer thereof, wherein patient's loss of cortical volume is reduced and the patient's MS-related dysfunction is reduced.

The present invention is further directed to a method of treating multiple sclerosis in a subject comprising administering to the subject, at regular dosing intervals, a pharmaceutical composition comprising a therapeutically effective amount of a compound of formula (I), or a pharmaceutically acceptable salt, prodrug, or isomer thereof, wherein patient's loss of cortical volume is reduced and the number of CE lesions in the patient is reduced.

The present invention is further directed to a method of treating multiple sclerosis in a subject comprising administering to the subject, at regular dosing intervals, a pharmaceutical composition comprising a therapeutically effective amount of a compound of formula (I), or a pharmaceutically acceptable salt, prodrug, or isomer thereof, wherein the subject has fewer CE lesions or T2 lesions than a subject not administered a pharmaceutical composition comprising a therapeutically effective amount of a compound of formula (I).

In one embodiment, the subject administered a pharmaceutical composition comprising a therapeutically effective amount of a compound of formula (I) has fewer CE lesions and T2 lesions than a subject not administered a pharmaceutical composition comprising a therapeutically effective amount of a compound of formula (I).

In any one of the above embodiments, the multiple sclerosis may be relapsing remitting multiple sclerosis.

In any one of the above embodiments, the compound of formula (I) may be in the form of a besylate salt.

In any one of the above embodiments, the regular dosing interval may be once daily.

In any one of the above embodiments, the therapeutically effective amount may be greater than about 1 milligram, about at least 3 milligram, about at least 5 milligram, from about 1 to about 3 milligram, from about 3 to about 10 milligrams, from about 3 to about 5 milligrams, about 3 milligrams, about 4 milligrams, about 5 milligrams, from about 5 to about 7 milligrams, from about 5 to about 10 milligrams, about 6 milligrams, about 7 milligrams, 8 milligrams, about 9 milligrams or about 10 milligrams.

In any one of the above embodiments, the method may reduce MS-related dysfunction.

In any one of the above embodiments, the MS-related dysfunction may be determined by Expanded Disability Status Scale (EDSS) or Multiple Sclerosis Functional Composite (MSFC).

### BRIEF DESCRIPTION OF THE DRAWINGS

FIG. 1. CE lesions in subjects at baseline.

FIG. 2. Reduction of cumulative CE lesions at 6 months.

FIG. 3. Reduction of new or enlarged T2 lesions at 6 months.

FIG. 4. Protection against whole brain volume loss at 6 months.

FIG. 5. Change in cortical volume and EDSS scores at 3 months.

FIG. 6. Change in cortical volume and EDSS scores at 6 months.

FIG. 7. Percent change in neocortical volume from baseline at month 6 vs. total new GAD CE T1 lesions over 6 months.

### DETAILED DESCRIPTION OF THE INVENTION

### Abbreviations and Definitions

The abbreviations used herein are conventional, unless otherwise defined.

The terms CHS-131 or INT-131 refer to the compound of formula (I)

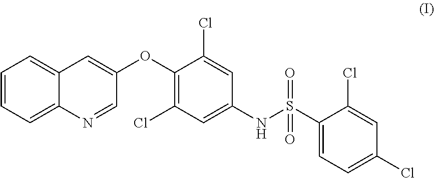 [SMILES: O=S(=O)(Nc1cc(Cl)c(Oc2cnc3ccccc3c2)c(Cl)c1)c1ccc(Cl)cc1Cl]

CHS-131 has been previously disclosed and characterized in U.S. Pat. Nos. 7,041,691; 6,200,995; 6,583,157 and 6,653,332, which are incorporated herein by reference in their entirety.

The terms “treat”, “treating” and “treatment” refer to a method of alleviating or abrogating a disease and/or its attendant symptoms.

The terms “prevent”, “preventing” and “prevention” refer to a method of decreasing the probability or eliminating the possibility that a disease will be contracted.

The term “therapeutically effective amount” refers to that amount of the compound being administered sufficient to prevent development of or alleviate to some extent one or more of the symptoms of the condition or disorder being treated.

The term “subject” is defined herein to include animals such as mammals, including but not limited to, primates (e.g., humans), cows, sheep, goats, horses, dogs, cats, rabbits, rats, mice and the like. In preferred embodiments, the subject is a human.

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either net or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either net or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isbutyric, oxalic, maleic, malonic, benzoic, succinic, suberic, fumeric mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al., “Pharmaceutical Salts”, Journal of Pharmaceutical Science, 1977, 66, 1-19). Certain specific compounds of the present inventions contain both basic and acidic functionalities that allow the compounds to be converted into either base or acid addition salts.

The neutral forms of the compounds may be registered by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In additional to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent. Prodrugs are often useful because, in some situations, they may be easier to administer than the parent drug. They may, be bioavailable by oral administration whereas the parent drug is not. The prodrug may also have improved solubility in pharmacological compositions over the parent drug. A wide variety of prodrug derivatives are known in the art, such as those that rely on hydrolytic cleavage or oxidative activation of the prodrug. An example, without limitation, of a prodrug would be a compound of the present invention which is administered as an ester (the “prodrug”), but then is metabolically hydrolyzed to the carboxylic acid, the active entity. Additional examples include peptidyl derivatives of a compound of the invention.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are all intended to be encompassed within the scope of the present invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

The present invention also includes methods of treating subjects in need thereof with a pharmaceutical composition comprising CHS-131 suitable for administration to the subject. Pharmaceutical compositions of CHS-131 suitable for use in the methods of the present invention include, but are not limited to, solid, gel, and liquid compositions comprising CHS-131. Pharmaceutical compositions may be administered, for example, topical administration, oral administration, or parenteral administration (e.g. subcutaneous, intravenous). Specific routes of administration within these classifications are known to one of skill in the art. Non-limiting examples of CHS-131 formulations are disclosed in U.S. Pat. No. 9,675,603 which is incorporated herein by reference in its entirety.

### Embodiments of the Invention

The present invention is directed to a method of reducing cortical atrophy or loss of cortical volume in a subject suffering from multiple sclerosis comprising administering to the subject, at regular dosing intervals, a pharmaceutical composition comprising a therapeutically effective amount of a compound of formula (I),

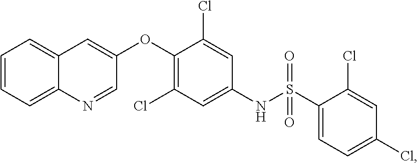 [SMILES: O=S(=O)(Nc1cc(Cl)c(Oc2cnc3ccccc3c2)c(Cl)c1)c1ccc(Cl)cc1Cl]

or a pharmaceutically acceptable salt, prodrug, or isomer thereof.

In one embodiment, the pharmaceutical composition is administered to the subject daily and the therapeutically effective amount of the compound is about 3 milligrams.

The present invention is further directed to a method of reducing loss of cortical volume in a subject suffering from multiple sclerosis comprising administering to the subject, at regular dosing intervals, a pharmaceutical composition comprising a therapeutically effective amount of a compound of formula (I), or a pharmaceutically acceptable salt, prodrug, or isomer thereof.

In one embodiment, the pharmaceutical composition is administered to the subject daily and the therapeutically effective amount of the compound is about 3 milligrams.

The present invention is further directed to a method of treating multiple sclerosis in a woman comprising administering to a woman at regular dosing intervals a pharmaceutical composition comprising a therapeutically effective amount of a compound of formula (I),

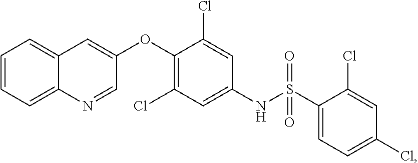 [SMILES: O=S(=O)(Nc1cc(Cl)c(Oc2cnc3ccccc3c2)c(Cl)c1)c1ccc(Cl)cc1Cl]

or a pharmaceutically acceptable salt, prodrug, or isomer thereof.

In one embodiment, the pharmaceutical composition is administered to the woman daily and the therapeutically effective amount of the compound is about 5 milligrams.

In one embodiment, the method provides a reduction in number of new gadolinium CE T1-weighted lesions in the woman over six months by at least about 45%, at least about 50%, at least about 60%, at least about 65%, at least about 70%, or at least about 80%.

The present invention is further directed to a method of treating multiple sclerosis in a subject comprising administering to the subject, at regular dosing intervals, a pharmaceutical composition comprising a therapeutically effective amount of a compound of formula (I), or a pharmaceutically acceptable salt, prodrug, or isomer thereof, wherein patient's loss of cortical volume is reduced and the patient's MS-related dysfunction is reduced.

The present invention is further directed to a method of treating multiple sclerosis in a subject comprising administering to the subject, at regular dosing intervals, a pharmaceutical composition comprising a therapeutically effective amount of a compound of formula (I), or a pharmaceutically acceptable salt, prodrug, or isomer thereof, wherein patient's loss of cortical volume is reduced and the number of CE lesions in the patient is reduced.

The present invention is further directed to a method of treating multiple sclerosis in a subject comprising administering to the subject, at regular dosing intervals, a pharmaceutical composition comprising a therapeutically effective amount of a compound of formula (I), or a pharmaceutically acceptable salt, prodrug, or isomer thereof, wherein the subject has fewer CE lesions or T2 lesions than a subject not administered a pharmaceutical composition comprising a therapeutically effective amount of a compound of formula (I).

In one embodiment, the subject administered a pharmaceutical composition comprising a therapeutically effective amount of a compound of formula (I) has fewer CE lesions and T2 lesions than a subject not administered a pharmaceutical composition comprising a therapeutically effective amount of a compound of formula (I).

In any one of the above embodiments, the multiple sclerosis may be relapsing remitting multiple sclerosis.

In any one of the above embodiments, the compound of formula (I) may be in the form of a besylate salt.

In any one of the above embodiments, the regular dosing interval may be once daily.

In any one of the above embodiments, the therapeutically effective amount may be greater than about 1 milligram, about at least 3 milligram, about at least 5 milligram, from about 1 to about 3 milligram, from about 3 to about 10 milligrams, from about 3 to about 5 milligrams, about 3 milligrams, about 4 milligrams, about 5 milligrams, from about 5 to about 7 milligrams, from about 5 to about 10 milligrams, about 6 milligrams, about 7 milligrams, 8 milligrams, about 9 milligrams or about 10 milligrams.

In any one of the above embodiments, the method may reduce MS-related dysfunction.

In any one of the above embodiments, the MS-related dysfunction may be determined by Expanded Disability Status Scale (EDSS) or Multiple Sclerosis Functional Composite (MSFC).

The invention will now be further described by the following non-limiting Examples.

### EXAMPLES

### Example 1-Phase 2 Study of Patients with Multiple Sclerosis

A Phase 2, randomized, double-blind, placebo-controlled, multi-center clinical trial of CHS-131 for the treatment of multiple sclerosis (MS) was conducted. The primary endpoint was the number of new gadolinium contrast-enhancing (CE) T1-weighted lesions on monthly MRI over 6 months.

The study enrolled MS patients in the relapsing and remitting course of the disease (RRMS) who had been diagnosed within ≤3 years of enrollment. Patients had to be treatment-naïve, have ≥1 gadolinium-positive lesion within 12 months of enrollment, and have an Expanded Disability Status Score (EDSS) of 0-6 at screening.

Part 1 was a double-blind, parallel-group, 6-month study. Patients were randomized to oral CHS-131 at 3 mg or 1 mg or placebo in a 1:1:1 ratio at 21 sites in Russia. Monthly MRIs were read in a blinded fashion at the Buffalo Neuroimaging and Analysis Center, Buffalo, N.Y., USA. Part 2 is an open label, 6-month, safety extension study, in which all subjects transition to 1 mg CHS-131 daily, to evaluate clinical response, CE lesions on MM, and safety.

Patient disposition and baseline characteristics are summarized in Tables 1 and 2 below.

TABLE 1
Disposition

| | 3 mg | 1 mg | |
|---|---|---|---|
| | CHS-131 | CHS-131 | Placebo |
| Randomized & Dosed | 76 | 76 | 75 |
| Complete efficacy | 70 | 70 | 69 |
| data in Part 1 | | | |
| Completed Part 1 | 73 | 74 | 72 |
| | (96.1%) | (97.4%) | (96.0%) |
| Discontinued | 3 | 2 | 3 |
| | (3.9%) | (2.6%) | (4.0%) |
| Pregnancy or lack | 1 | 0 | 1 |
| of birth control | | | |
| Withdrew consent | 2 | 1 | 1 |
| Lost to follow up | 0 | 1 | 0 |
| Other | 0 | 0 | 1 |

TABLE 2
Patient Characteristics at Baseline

| | 3 mg | 1 mg | |
|---|---|---|---|
| | CHS-131 | CHS-131 | Placebo |
| Randomized & Dosed | 76 | 76 | 75 |
| Female: | 60.5% | 60.5% | 74.3% |
| Caucasian: | 100% | 100% | 100% |
| Mean Age (years) | 30.5 | 30.8 | 31.9 |
| Age Range (years) | 19-47 | 19-49 | 19-50 |
| Mean Body-Mass | 23.4 | 23.5 | 24.2 |
| Index (kg/m²) | | | |
| Mean EDSS | 2.2 | 2.0 | 2.1 |

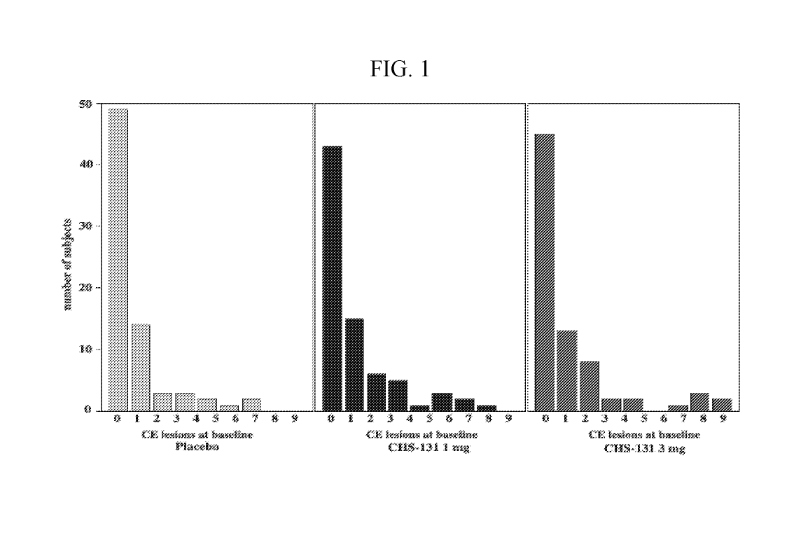

There were no significant differences in the baseline characteristics among the 3 mg, 1 mg and placebo groups. FIG. 1 demonstrates that the groups were similar in the distribution of number of subjects with numbers of CE lesions. Groups were also similar at baseline in age, gender, body mass index (BMI), EDSS, and disease duration.

### Phase 2 Study Results

### Contrast-Enhancing Lesions

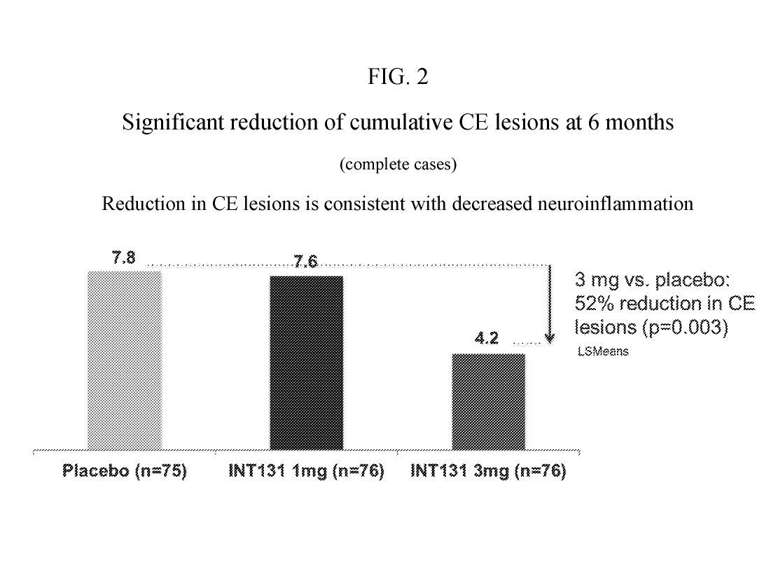

Treatment with CHS-131 resulted in a reduction of CE lesions. The mean cumulative number of new CE lesions over 6 months was 4.2 (LSMean 3.10) for 3 mg CHS131 (n=70), 7.6 (LSMean 5.15) for 1 mg CHS131 (n=70), and 7.8 (LSMean 6.49) for placebo (n=69). The response was dose-dependent. Based on appropriate statistical modeling (e.g. patients with complete efficacy data), the incidence of new CE lesions with 3 mg CHS-131 was significantly lower (52% reduction) than with placebo (p=0.003), and the incidence with 1 mg CHS-131 was 21% lower than with placebo. FIG. 2 provides a graphical representation of these LSMean data.

### T2 Lesions

Treatment with CHS-131 Resulted in a Reduction of New and Enlarged T2 Lesions.

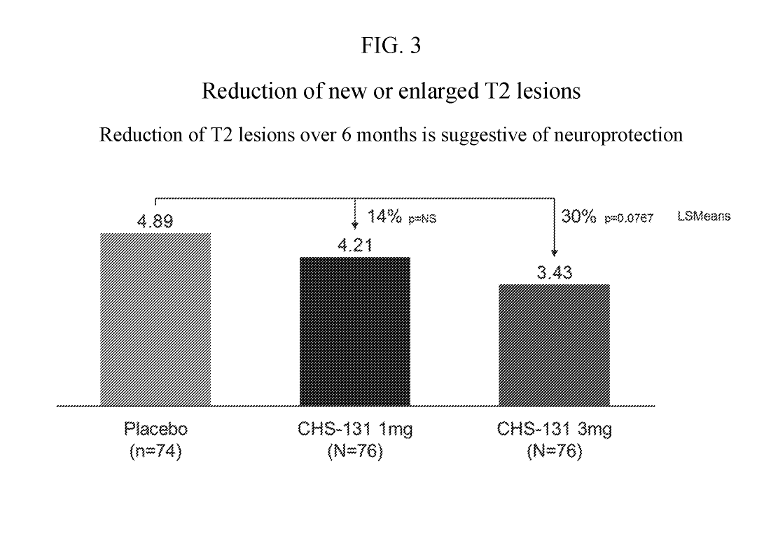

The mean number of new and enlarged lesions over 6 months was 3.43 for 3 mg CHS131 (n=76), 4.21 for 1 mg CHS131 (n=76), and 4.89 for placebo (n=74). The response was dose-dependent. Based on LSMeans analysis, treatment with 3 mg CHS-131 resulted in a 30% reduction in T2 lesions compared to placebo (p=0.0767). Treatment with 1 mg CHS-131 resulted in a 14% reduction in T2 lesions compared to placebo. FIG. 3 provides a graphical representation of these data.

Reduction of T2 lesions indicates that treatment with CHS-131 provides neuroprotection.

### Expanded Disability Status Scale (EDSS) Score

The EDSS scores are reported for the full analysis population in the table below.

TABLE 3
EDSS Scores

| | | CHS-131 | CHS-131 | Placebo |
|---|---|---|---|---|
| | | 3 mg (N = 76) | 1 mg (N = 76) | (N = 74) |
| Baseline | n | 76 | 76 | 74 |
| EDSS | Mean | 2.18 (0.851) | 2.04 (0.944) | 2.12 (0.823) |
| | (SD) | | | |
| Month 3 | n | 74 | 74 | 74 |
| EDSS | Mean | 2.17 (0.945) | 2.03 (0.961) | 2.15 (0.913) |
| | (SD) | | | |
| | % change | −0.46% | −0.49% | 1.42% |
| | from | | | |
| | baseline | | | |
| Month 6 | n | 73 | 74 | 73 |
| EDSS | Mean | 2.08 (0.846) | 1.98 (0.974) | 2.19 (0.949) |
| | (SD) | | | |
| | % change | −4.59% | −2.94% | 3.30% |
| | from | | | |
| | baseline | | | |

EDSS scores for patients receiving placebo increased over time. However, patients who received either 1 mg or 3 mg daily of CHS-131 had reduced EDSS scores at the 3 month and 6 month timepoints. This indicates MS patients who take CHS-131 have reduction in dysfunction.

### Annualized Confirmed Relapse Rate (ARR)

The ARR was lower for both 3 mg CHS-131 (0.26) and 1 mg (0.28) as compared with placebo (0.35). Thus, administering CHS-131 reduces the rate at which relapses of worsening neurological function (e.g. flare-ups or exacerbations) occur. Put otherwise, administration of CHS-131 increases the time between relapses.

### Safety

The most common (<2% overall) Treatment Emergent Adverse events (AE) were respiratory tract infection, respiratory tract infection, and headache. In the first six months of the study, AE's were reported in 34.2%, 26.3%, and 37.3% of patients in the 3 mg CHS-131, 1 mg CHS-131, and placebo groups, respectively. Treatment related AE's were reported in 10.5%, 3.9%, and 8.0% of patients in the 3 mg CHS-131, 1 mg CHS-131, and placebo groups, respectively. One subject was discontinued due to elevated liver function tests (LFTs) over the 12 months of the study. No new safety signals were detected for CHS-131. There was no evidence of the immunosuppression, cardiovascular or other common toxicities observed with PPARγ full agonists.

### Conclusions

This study demonstrated a statistically-significant decrease in the incidence of new CE lesions with 3 mg CHS-131 as compared with placebo over 6 months. Administration of CHS-131 also resulted in a reduction of new and enlarged T2 lesions. Additionally, CHS-131 reduced the AAR in MS patients. CHS-131 was generally well-tolerated with no evidence of immunosuppression.

### Example 2. Sex Based Dosing of CHS-131

Upon completion of the six month trial period described in Example 1, the number of new gadolinium CE T1-weighted lesions was evaluated in the entire study population and separately in men and women. Table 4 below reports all observed data from the study for new gadolinium CE T1-weighted lesions.

TABLE 4
Mean Number of New Gadolinium CE T1-weighted Lesions
on Monthly MRI over 6 Months by Dose Group

| | Male & Female | | Male | | Female | |
|---|---|---|---|---|---|---|
| | | Percent | | Percent | | Percent |
| | | Change | | Change | | Change |
| Dose | Mean # | from | Mean # | from | Mean # | from |
| (mg) | of Lesions | Placebo | of Lesions | Placebo | of Lesions | Placebo |
| 0 | 7.96 | | 8.53 | | 7.76 | |
| 1 | 7.19 | −9.6% | 5.93 | −30.4% | 8.01 | 3.2% |
| 3 | 4.51 | −43.3% | 3.81 | −55.4% | 4.97 | −36.0% |

The ratios of changes in lesions were evaluated by a least square means (LSMeans) analysis. In such an analysis, multiple factors including both categorical (e.g. treatment, center, gender) and continuous covariates (e.g. baseline measures) can be accounted for.

TABLE 5
Least-Square means analysis of Mean Number of New Gadolinium CE
T1-weighted Lesions on Monthly MRI over 6 Months by Dose Group

| | Male & Female | | Male | | Female | |
|---|---|---|---|---|---|---|
| | | Percent Change | | Percent Change | | Percent Change |
| Dose | LSMean # | from Placebo* | LSMean # | from Placebo* | LSMean # | from Placebo* |
| (mg) | of Lesions | (p value) | of Lesions | (p value) | of Lesions | (p value) |
| 0 | 6.49 | | 8.35 | | 5.79 | |
| 1 | 5.15 | −21% (0.3049) | 4.31 | −48% (0.0827) | 5.48 | −5% (0.8481) |
| 3 | 3.10 | −52% (0.0016) | 2.42 | −71% (0.0012) | 4.18 | −28% (0.2516) |

*Inverse of the ratio of LSMeans for treatment:placebo.

These data show that overall, the doses studied reduced the number of new T1 lesions, with the 3 mg dose resulting in a statistically significant 53% reduction in the whole population.

The data also show that males had better responses to 1 mg and 3 mg doses than females did. That is, there was a greater reduction in the mean number of new gadolinium CE T1-weighted lesions in men, whether taking 1 mg or 3 mg of CHS-131 daily. The 1 mg daily of CHS-131 did not result in a substantial reduction in the average number of new gadolinium CE T1-weighted lesions for women. However, women taking 3 mg of CHS-131 exhibited a substantial reduction in lesions.

Since a dose dependent response is seen for CHS-131 and the female subjects show a response at the higher studied dose, it is expected that a dose greater than 3 mg per day of CHS-131 will provide greater therapeutic benefit for females.

Analysis

To test the hypothesis that women will benefit from higher doses of CHS-131 and identify efficacious doses of CHS-131 for women, a dose response curve was calculated. The dose response curve was calculated to determine the efficacy of various daily dose of CHS-131 for males and females. The dose-response curve is assumed to have the following form:

RDose=α exp β−Dose+γ

Parameter β was determined from negative binomial regression using the observed total new GAD CE lesions 6 Months and numeric dosage of 0, 1, 3 mg. Parameters α and γ were determined using least-squares method on the observed mean total new GAD CE lesions 6 Months.

TABLE 6
Dose response curve parameters

| Parameter | Overall | Male | Female |
|---|---|---|---|
| α | 7.94 | 8.66 | 7.92 |
| β | −0.20 | −0.26 | −0.16 |
| γ | 0.26 | −0.36 | 0.38 |

Based on the clinical trial data, we calculated the expected number of new gadolinium CE T1-weighted lesions that would be seen over six months on doses of CHS-131 ranging from 1 to 10 mg per day. Table 7 below provides a tabular report of the percent reduction compared to the number of lesions at a 0 mg dose.

TABLE 7
Expected reduction of new gadolinium CE T1-weighted
lesions over six months on doses of CHS-131 as compared
to the number of lesions at a 0 mg dose.

| CHS- | | | | | | |
|---|---|---|---|---|---|---|
| 131 | Males & Females | | Males | | Females | |
| Dose | # of | Percent | # of | Percent | # of | Percent |
| (mg) | Lesions | Reduction | Lesions | Reduction | Lesions | Reduction |
| 0 | 8.20 | 0.00% | 8.30 | 0.00% | 8.30 | 0.00% |
| 1 | 6.79 | 17.20% | 6.33 | 23.73% | 7.14 | 13.98% |
| 2 | 5.63 | 31.34% | 4.81 | 42.05% | 6.15 | 25.90% |
| 3 | 4.67 | 43.05% | 3.64 | 56.14% | 5.31 | 36.02% |
| 4 | 3.89 | 52.56% | 2.73 | 67.11% | 4.59 | 44.70% |
| 5 | 3.24 | 60.49% | 2.03 | 75.54% | 3.97 | 52.17% |
| 6 | 2.71 | 66.95% | 1.49 | 82.05% | 3.45 | 58.43% |
| 7 | 2.27 | 72.32% | 1.07 | 87.11% | 3.00 | 63.86% |
| 8 | 1.92 | 76.59% | 0.74 | 91.08% | 2.62 | 68.43% |
| 9 | 1.62 | 80.24% | 0.49 | 94.10% | 2.29 | 72.41% |
| 10 | 1.38 | 83.17% | 0.30 | 96.39% | 2.01 | 75.78% |

These data show that increasing doses will reduce the number of new lesions in MS patients. Table 7 does not account for any placebo effect and shows the relative reduction in a treatment group. Based on these data, a 3 mg per day dose in men, which was statistically significant in the observed data, will have a 56% reduction in the number of new lesions. In women, Table 7 shows a 5 mg per day dose results in a 52% reduction in the number of new lesions which is similar to the 3 mg per day dose in men. Thus, it is expected that at least 5 mg per day of CHS-131 will treat women with MS.

To account for placebo effect, Table 8 shows the percent change in the number of new gadolinium CE T1-weighted lesions over six months on doses of CHS-131.

TABLE 8
Expected reduction compared to placebo group of new gadolinium
CE Tl- weighted lesions over six months on doses of CHS-131

| | Males & Females | | Males | | Females | |
|---|---|---|---|---|---|---|
| INT- | | Percent | | Percent | | Percent |
| 131 | | Change | | Change | | Change |
| Dose | # of | from | # of | from | # of | from |
| (mg) | Lesions | Placebo | Lesions | Placebo | Lesions | Placebo |
| 0 | 8.20 | −17.2% | 8.30 | −23.7% | 8.30 | −13.9% |
| 1 | 6.79 | −31.4% | 6.33 | −42.0% | 7.14 | −25.9% |
| 2 | 5.63 | −43.0% | 4.81 | −56.1% | 6.15 | −36.0% |
| 3 | 4.67 | −52.6% | 3.64 | −67.1% | 5.31 | −44.7% |
| 4 | 3.89 | −60.5% | 2.73 | −75.5% | 4.59 | −52.1% |
| 5 | 3.24 | −66.9% | 2.03 | −82.1% | 3.97 | −58.5% |
| 6 | 2.71 | −72.3% | 1.49 | −87.1% | 3.45 | −63.9% |
| 7 | 2.27 | −76.6% | 1.07 | −91.0% | 3.00 | −68.5% |
| 8 | 1.92 | −80.2% | 0.74 | −94.1% | 2.62 | −72.4% |
| 9 | 1.62 | −83.2% | 0.49 | −96.4% | 2.29 | −75.8% |
| 10 | 1.38 | −100.0% | 0.30 | −100.0% | 2.01 | −100.0% |

Even when a placebo effect is taken into account, the results shown in Table 8 indicate that a 5 mg per day does in women will result in a 56% reduction in lesions as compared to placebo. This approaches the reduction calculated for men taking the 5 mg per day dose (67%) which resulted in a statistically significant reduction in the observed data.

The statistically significant 71% reduction of new T1 lesions in men from a 3 mg per day dose of CHS-131 in the study analysis aligns to a 67% reduction in new lesions on the dose response curve in Table 8. Based on the similarity of these values, the dose response curve provides a good means for establishing the effectiveness of doses of CHS-131. Further, CHS-131 has been reported in clinical studies to be safe. Therefore, it is expected that subjects can safely be administered doses of CHS-131 greater than 3 mg per day to achieve improved therapeutic benefit.

Since 3 mg of CHS-131 provided a statistically significant reduction of new lesions in men, a similar percent reduction of new lesions in women is expected to correlate with an efficacious dose of CHS-131. Therefore, based on the dose response curve, at least 5 mg per day of CHS-131 will treat MS in women. The dose response curve also supports the conclusion that CHS-131 doses of at least 6 mg per day, 7 mg per day, 8 mg per day, 9 mg per day, and 10 mg per day will be effective in treating MS in women.

### Example 3. CHS-131 Reduces Loss of Cortical and Whole Brain Volume

Upon completion of the six-month trial period described in Example 1 the change in neocortical volume and whole brain volume was evaluated.

Table 9 below reports the change in neocortical volumes at 3 months and at 6 months, compared to baseline, for the 3 mg and placebo groups.

### Cortical Volume

Daily treatment with CHS-131 protects against loss of cortical volume.

TABLE 9
Change in cortical volume over 3 months and 6 months

| | | CHS-131 | CHS-131 | |
|---|---|---|---|---|
| | | 3 mg | 1 mg | Placebo |
| | | (N = 76) | (N = 76) | (N = 74) |
| Percent change | n | 61 | 57 | 57 |
| from baseline to 3 | Mean | −0.297 | −0.706 | −0.517 |
| month | (SD) | (1.4133) | (1.5465) | (1.1400) |
| | Median | −0.325 | −0.746 | −0.449 |
| Percent change | n | 56 | 50 | 45 |
| from baseline to 6 | Mean | −0.709 | −1.350 | −1.077 |
| month | (SD) | (1.4809) | (1.6398) | (1.2227) |
| | Median | −0.832 | −1.428 | −1.144 |

Baseline volume was normalized for head size. Percent change is calculated by SIENAX-multi-time point (MTP3) algorithm. The difference in the mean change in volume calculated to determine the impact treatment with CHS-131. The results are reported in Table 10, below.

TABLE 10
Difference in cortical volume at 3 months and 6 months

| | CHS-131 3 mg | Placebo | Reduction in |
|---|---|---|---|
| | Cortical | Cortical | cortical |
| | volume rate | volume rate | volume loss |
| | of loss | of loss | from CHS-131 |
| 3 Months | −0.297 | −0.517 | 42.6% |
| 6 Months | −0.709 | −1.077 | 34.2% |

Surprisingly, these data show that treatment with 3 mg of CHS-131 reduced the loss of cortical volume by 42.6% at 3 months and 34.2% at 6 months, as compared to placebo. No reduction in the loss of cortical volume was observed in patients treated with 1 mg of CHS-131.

This result is unexpected since 1 mg of CHS-131 reduced the number of CE lesions compared to placebo, as did the 3 mg dose. It is therefore surprising that only the 3 mg daily dose of CHS-131 reduced the loss of cortical volume in MS patients. Thus, greater than 1 mg daily dose of CHS-131 was required to reduce the loss of cortical volume and a dose of at least 3 mg daily showed a reduction in the loss of cortical volume.

### Whole Brain Volume

Whole brain volume was evaluated at baseline and six months in this study. Neural atrophy was measured by serial determination of brain volume using serial MRIs.

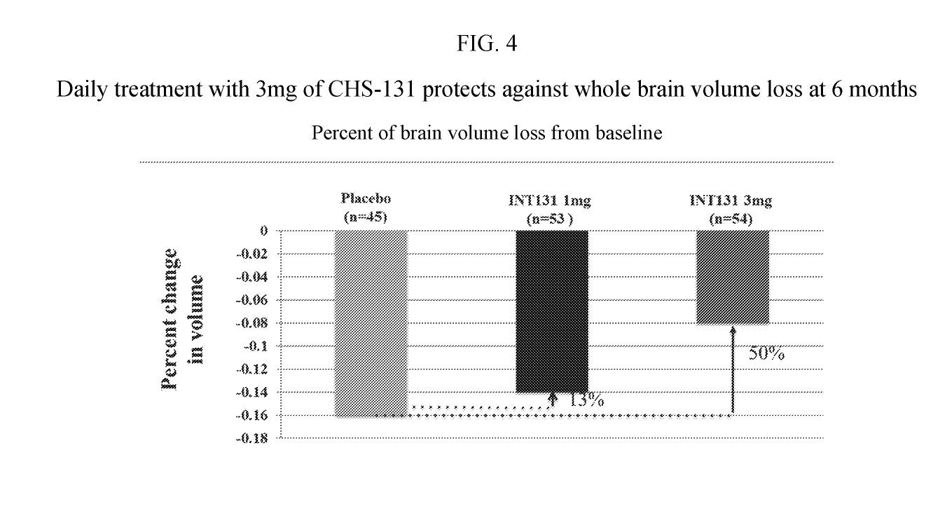

Patients treated daily with 1 mg CHS-131 daily had a 13% less neural volume loss, compared to placebo. In contrast, patients treated daily with 3 mg of CHS-131 had 50% less parenchymal atrophy than the placebo treated cohort. In real terms, the placebo group lost 0.16% and the 1 mg/day CHS-131 treatment cohort lost 0.14% of their brain volumes over 6 months, respectively, while the cohort treated with 3 mg/day of CHS-131 lost 0.08% of their neural parenchyma volume over the same time course. FIG. 4 provides a graphical representation of these data. The extent of neural atrophy in the placebo group is consistent with reported brain atrophy seen in RRMS patients.

These data indicate that daily, oral treatment with CHS-131 protects against neural atrophy. Even though CHS-131 lacks the TZD (glitazone) scaffold of other PPARγ agonists (e.g. rosiglitazone and pioglitazone) and selectively activates AF2, the neural protection observed in the cohort treated with daily, oral 3 mg data is consistent with published reports demonstrating the neural protective activities of PPARγ agonists. Thus, treatment with CHS-131 should protect against neural atrophy without the adverse events commonly seen in TZD treatments.

### Example 4. Loss of Cortical Volume and EDSS Score

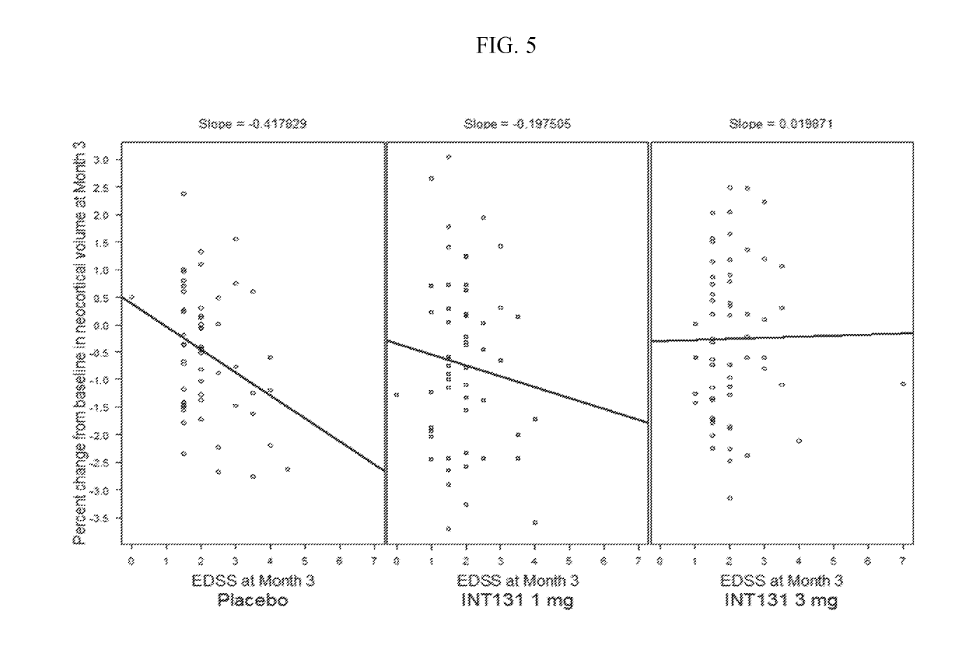

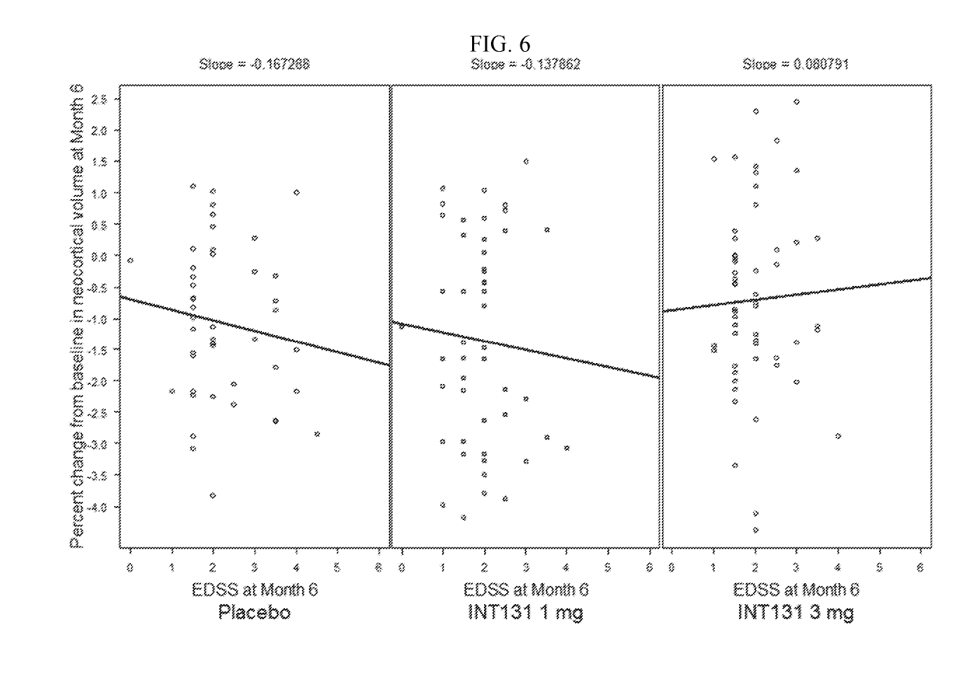

To evaluate the connection between cortical volume loss and dysfunction, the reduction in volume loss was compared to EDSS scores of the MS patients. FIGS. 5 and 6 show the relationship between the change in cortical volume and EDSS scores at 3 months and 6 months, respectively.

At the 3-month time point (FIG. 5), the trend line indicates patients taking 3 mg of CHS-131 show reduction in loss of cortical volume across all EDSS scores, while higher loss of cortical volume was observed in placebo patients with increased EDSS score. Likewise, at the 6-month time point (FIG. 6), the trend line indicates patients taking 3 mg of CHS-131 show reduction in loss of cortical volume across all EDSS scores, while higher loss of cortical volume was observed in placebo patients with increased EDSS score.

At the 6-month time point there is little difference between the CHS-131 1 mg does and placebo trend lines—slopes of −0.138 and −0.167, respectively. However, CHS-131 3 mg dose has a trend line slope of 0.080. This is surprising since the at 3-month time point, the CHS-131 1 mg dose trend line showed improvement over the placebo trend line. These results indicate that patients who take 3 mg of CHS-131 per day have reduction in loss of cortical volume across all EDSS scores. These results demonstrate that CHS-131 imparts physiological improvement to patients (i.e. reduction in cortical volume loss) and improvement in function (i.e. reduced dysfunction as measured by EDSS—improved or non-increasing EDSS scores).

Based on these results, a daily dose of at least 3 mg of CHS-131 reduces cortical volume loss in patients with multiple sclerosis. Also, patients taking at least 3 mg of CHS-131 have less clinical dysfunction. Reduced clinical dysfunction, or increased function, is shown by an improvement in disability scores (e.g. EDSS and MSFC). Patients taking 3 mg of CHS-131 show reduction in loss of cortical volume across all EDSS scores, while higher loss of cortical volume was observed in placebo patients with increased EDSS score. It is therefore expected that individuals with multiple sclerosis that take a daily dose of at least 3 mg of CHS-131 will have non-increasing EDSS or MSFC scores or improved EDSS or MSFC scores. Daily CHS-131 doses of greater than 1 mg, at least 3 mg, 4 mg, 5 mg, 6 mg, 7 mg, 8 mg, 9 mg, and 10 mg will be effective reducing the loss of cortical volume and improving EDSS or MSFC scores.

### Example 5. Reduction in Cortical Volume Loss and CE Lesions

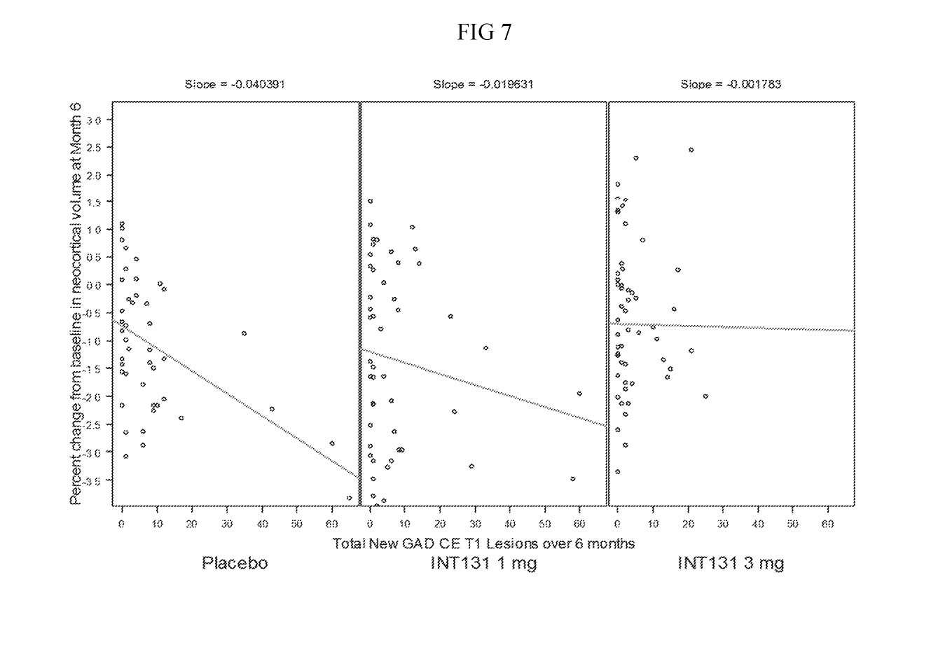

To evaluate the connection between the reduction in cortical volume loss and CHS-131 treatment, cortical volume was compared to observed CE lesion in the MS patients. FIG. 7 shows the percent change in neocortical volume from baseline at month 6 vs. total new GAD CE T1 lesions over 6 months in complete cases.

The slopes of cortical atrophy at six months as a function of CE lesion number at six months was plotted for the placebo group (n=69), the CHS-131 1 mg/day group (n=70), and the CHS-131 3 mg/day group (n=70). For the placebo, 1 mg/day, and 3 mg/day groups the slopes were: −0.040391, −0.019631, and −0.001783, respectively. Comparing the slopes of cortical atrophy as a function of CE lesion number demonstrates a clear, dose-dependent effect of CHS-131 on the sparing of neocortical volume. Thus, the reduction in CE lesions as a result of CHS-131 treatment is correlated with a reduction in the loss of cortical volume in MS patients.

Based on these results, a daily dose of at least 3 mg of CHS-131 reduces cortical volume loss in patents with multiple sclerosis. Since the reduction is dose dependent, daily CHS-131 doses of at least 3 mg, 4 mg, 5 mg, 6 mg, 7 mg, 8 mg, 9 mg, and 10 mg will be effective reducing the loss of cortical volume and reducing the number of CE lesions in MS patients.

What is claimed is:

1. A method of treating multiple sclerosis in a woman comprising administering to a woman at regular dosing intervals a pharmaceutical composition comprising a therapeutically effective amount of a compound of formula (I), 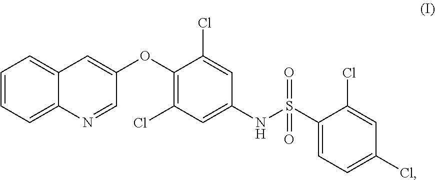 [SMILES: O=S(=O)(Nc1cc(Cl)c(Oc2cnc3ccccc3c2)c(Cl)c1)c1ccc(Cl)cc1Cl]
or a pharmaceutically acceptable salt, prodrug, or isomer thereof.

2. The method of claim 1, wherein the multiple sclerosis is relapsing remitting multiple sclerosis.

3. The method of claim 1, wherein the compound of formula (I) is in the form of a besylate salt.

4. The method of claim 1, wherein the regular dosing interval is once daily.

5. The method of claim 1, wherein the therapeutically effective amount is from about 5 to about 10 milligrams.

6. The method of claim 5, wherein the therapeutically effective amount is about 5 milligrams.

7. The method of claim 1, wherein the pharmaceutical composition is administered to the woman daily and the therapeutically effective amount of the compound is about 5 milligrams.

8. The method of claim 1, wherein the method provides a reduction in number of new gadolinium CE T1-weighted lesions in the woman over six months by at least about 45%, at least about 50%, at least about 60%, at least about 65%, at least about 70%, or at least about 80%.

9. A method of reducing cortical atrophy in a subject suffering from multiple sclerosis comprising administering to the subject, at regular dosing intervals, a pharmaceutical composition comprising a therapeutically effective amount of a compound of formula (I), 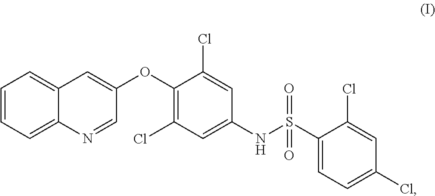 [SMILES: O=S(=O)(Nc1cc(Cl)c(Oc2cnc3ccccc3c2)c(Cl)c1)c1ccc(Cl)cc1Cl]
or a pharmaceutically acceptable salt, prodrug, or isomer thereof.

10. The method of claim 9, wherein the multiple sclerosis is relapsing remitting multiple sclerosis.

11. The method of claim 9, wherein the compound of formula (I) is in the form of a besylate salt.

12. The method of claim 9, wherein the regular dosing interval is once daily.

13. The method of claim 9, wherein the therapeutically effective amount is from about 3 to about 10 milligrams.

14. The method of claim 13, wherein the therapeutically effective amount is about 3 milligrams.

15. The method of claim 9, wherein the pharmaceutical composition is administered to the subject daily and the therapeutically effective amount of the compound is about 3 milligrams.

16. The method of claim 9, wherein the method reduces MS-related dysfunction.

17. The method of claim 16, wherein the MS-related dysfunction is determined by Expanded Disability Status Scale (EDSS) or Multiple Sclerosis Functional Composite (MSFC).

18. A method of reducing loss of cortical volume in a subject suffering from multiple sclerosis comprising administering to the subject, at regular dosing intervals, a pharmaceutical composition comprising a therapeutically effective amount of a compound of formula (I), 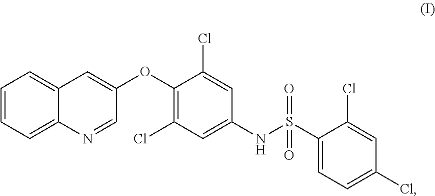 [SMILES: O=S(=O)(Nc1cc(Cl)c(Oc2cnc3ccccc3c2)c(Cl)c1)c1ccc(Cl)cc1Cl]
or a pharmaceutically acceptable salt, prodrug, or isomer thereof.

19. The method of claim 18, wherein the multiple sclerosis is relapsing remitting multiple sclerosis.

20. The method of claim 18, wherein the compound of formula (I) is in the form of a besylate salt.

21. The method of claim 18, wherein the regular dosing interval is once daily.

22. The method of claim 18, wherein the therapeutically effective amount is from about 3 to about 10 milligrams.

23. The method of claim 22, wherein the therapeutically effective amount is about 3 milligrams.

24. The method of claim 18, wherein the pharmaceutical composition is administered to the subject daily and the therapeutically effective amount of the compound is about 3 milligrams.

25. The method of claim 18, wherein the method reduces MS-related dysfunction.

26. The method of claim 25, wherein the MS-related dysfunction is determined by Expanded Disability Status Scale (EDSS) or Multiple Sclerosis Functional Composite (MSFC).

27. A method of treating multiple sclerosis in a subject comprising administering to the subject, at regular dosing intervals, a pharmaceutical composition comprising a therapeutically effective amount of a compound of formula (I), 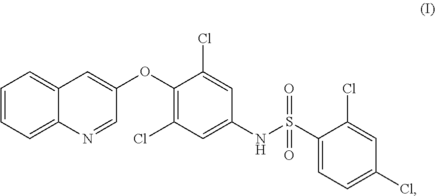 [SMILES: O=S(=O)(Nc1cc(Cl)c(Oc2cnc3ccccc3c2)c(Cl)c1)c1ccc(Cl)cc1Cl]
or a pharmaceutically acceptable salt, prodrug, or isomer thereof, wherein patient's loss of cortical volume is reduced and the patient's MS-related dysfunction is reduced.

28. The method of claim 27, wherein the multiple sclerosis is relapsing remitting multiple sclerosis.

29. The method of claim 27, wherein the compound of formula (I) is in the form of a besylate salt.

30. The method of claim 27, wherein the regular dosing interval is once daily.

31. The method of claim 27, wherein the therapeutically effective amount is from about 3 to about 10 milligrams.

32. The method of claim 31, wherein the therapeutically effective amount is about 3 milligrams.

33. The method of claim 27, wherein the pharmaceutical composition is administered to the subject daily and the therapeutically effective amount of the compound is about 3 milligrams.

34. The method of claim 27, wherein the MS-related dysfunction is determined by Expanded Disability Status Scale (EDSS) or Multiple Sclerosis Functional Composite (MSFC).

35. A method of treating multiple sclerosis in a subject comprising administering to the subject, at regular dosing intervals, a pharmaceutical composition comprising a therapeutically effective amount of a compound of formula (I), 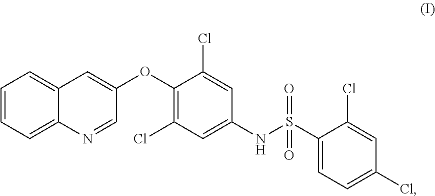 [SMILES: O=S(=O)(Nc1cc(Cl)c(Oc2cnc3ccccc3c2)c(Cl)c1)c1ccc(Cl)cc1Cl]
or a pharmaceutically acceptable salt, prodrug, or isomer thereof, wherein patient's loss of cortical volume is reduced and the number of CE lesions in the patient is reduced.

36. The method of claim 35, wherein the multiple sclerosis is relapsing remitting multiple sclerosis.

37. The method of claim 35, wherein the compound of formula (I) is in the form of a besylate salt.

38. The method of claim 35, wherein the regular dosing interval is once daily.

39. The method of claim 35, wherein the therapeutically effective amount is from about 3 to about 10 milligrams.

40. The method of claim 39, wherein the therapeutically effective amount is about 3 milligrams.

41. The method of claim 35, wherein the pharmaceutical composition is administered to the subject daily and the therapeutically effective amount of the compound is about 3 milligrams.

42. A method of treating multiple sclerosis in a subject comprising administering to the subject, at regular dosing intervals, a pharmaceutical composition comprising a therapeutically effective amount of a compound of formula (I), 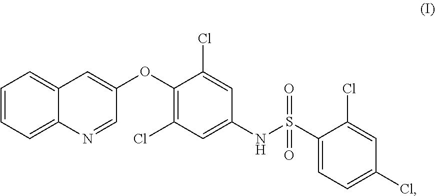 [SMILES: O=S(=O)(Nc1cc(Cl)c(Oc2cnc3ccccc3c2)c(Cl)c1)c1ccc(Cl)cc1Cl]
or a pharmaceutically acceptable salt, prodrug, or isomer thereof, wherein the subject has fewer CE lesions or T2 lesions than a subject not administered a pharmaceutical composition comprising a therapeutically effective amount of a compound of formula (I).

43. The method of claim 42, wherein the multiple sclerosis is relapsing remitting multiple sclerosis.

44. The method of claim 42, wherein the compound of formula (I) is in the form of a besylate salt.

45. The method of claim 42, wherein the regular dosing interval is once daily.

46. The method of claim 42, wherein the therapeutically effective amount is from about 3 to about 10 milligrams.

47. The method of claim 46, wherein the therapeutically effective amount is about 3 milligrams.

48. The method of claim 42, wherein the pharmaceutical composition is administered to the subject daily and the therapeutically effective amount of the compound is about 3 milligrams.

49. The method of claim 42, wherein the subject administered a pharmaceutical composition comprising a therapeutically effective amount of a compound of formula (I) has fewer CE lesions and T2 lesions than a subject not administered a pharmaceutical composition comprising a therapeutically effective amount of a compound of formula (I).
